SEQUENCE LISTING (2 sequences)

SEQ ID NO: 1 - protein, 1287 aa, Homo sapiens
MHPDLGPLCTLLYVTLTILCSSVSSDLAPYFTSEPLSAVQKLGGPVVLHCSAQPVTTRISWLHNGKTLDGNLEHVKIHQGTLTILSLNSSLLGYYQCLANNSIGAIVSGPATVSVAVLGDFGSSTKHVITAEEKSAGFIGCRVPESNPKAEVRYKIRGKWLEHSTENYLILPSGNLQILNVSLEDKGSYKCAAYNPVTHQLKVEPIGRKLLVSRPSSDDVHILHPTHSQALAVLSRSPVTLECVVSGVPAPQVYWLKDGQDIAPGSNWRRLYSHLATDSVDPADSGNYSCMAGNKSGDVKYVTYMVNVLEHASISKGLQDQIVSLGATVHFTCDVHGNPAPNCTWFHNAQPIHPSARHLTAGNGLKISGVTVEDVGMYQCVADNGIGFMHSTGRLEIENDGGFKPVIITAPVSAKVADGDFVTLSCNASGLPVPVIRWYDSHGLITSHPSQVLRSKSRKSQLSRPEGLNLEPVYFVLSQAGASSLHIQAVTQEHAGKYICEAANEHGTTQAEASLMVVPFETNTKAETVTLPDAAQNDDRSKRDGSETGLLSSFPVKVHPSAVESAPEKNASGISVPDAPIILSPPQTHTPDTYNLVWRAGKDGGLPINAYFVKYRKLDDGVGMLGSWHTVRVPGSENELHLAELEPSSLYEVLMVARSAAGEGQPAMLTFRTSKEKTASSKNTQASSPPVGIPKYPVVSEAANNNFGVVLTDSSRHSGVPEAPDRPTISTASETSVYVTWIPRANGGSPITAFKVEYKRMRTSNWLVAAEDIPPSKLSVEVRSLEPGSTYKFRVIAINHYGESFRSSASRPYQVVGFPNRFSSRPITGPHIAYTEAVSDTQIMLKWTYIPSSNNNTPIQGFYIYYRPTDSDNDSDYKRDVVEGSKQWHMIGHLQPETSYDIKMQCFNEGGESEFSNVMICETKVKRVPGASEYPVKDLSTPPNSLGSGGNVGPATSPARSSDMLYLIVGCVLGVMVLILMVFIAMCLWKNRQQNTIQKYDPPGYLYQGSDMNGQMVDYTTLSGASQINGNVHGGFLTNGGLSSGYSHLHHKVPNAVNGIVNGSLNGGLYSGHSNSLTRTHVDFEHPHHLVNGGGMYTAVPQIDPLECVNCRNCRNNNRCFTKTNSTFSSSPPPVVPVVAPYPQDGLEMKPLSHVKVPVCLTSAVPDCGQLPEESVKDNVEPVPTQRTCCQDIVNDVSSDGSEDPAEFSRGQEGMINLRIPDHLQLAKSCVWEGDSCAHSETEINIVSWNALILPPVPEGCAEKTMWSPPGIPLDSPTEVLQQPRET

SEQ ID NO: 2 - protein, 396 aa, Homo sapiens
MALLTNLLPLCCLALLALPAQSCGPGRGPVGRRRYARKQLVPLLYKQFVPGVPERTLGASGPAEGRVARGSERFRDLVPNYNPDIIFKDEENSGADRLMTERCKERVNALAIAVMNMWPGVRLRVTEGWDEDGHHAQDSLHYEGRALDITTSDRDRNKYGLLARLAVEAGFDWVYYESRNHVHVSVKADNSLAVRAGGCFPGNATVRLWSGERKGLRELHRGDWVLAADASGRVVPTPVLLFLDRDLQRRASFVAVETEWPPRKLLLTPWHLVFAARGPAPAPGDFAPVFARRLRAGDSVLAPGGDALRPARVARVAREEAVGVFAPLTAHGTLLVNDVLASCYAVLESHQWAHRAFAPLRLLHALGALLPGGAVQPTGMHWYSRLLYRLAEELLG

(19) United States
(12) Patent Application Publication
(10) Pub. No.: US 2023/0132275 A1
(43) Pub. Date: Apr. 27, 2023

(54) USE OF CDON INHIBITORS FOR THE TREATMENT OF ENDOTHELIAL DYSFUNCTION

(71) Applicants: INSERM (INSTITUT NATIONAL DE LA SANTÉ ET DE LA RECHERCHE MÉDICALE), Paris (FR); UNIVERSITÉ DE BORDEAUX, BORDEAUX (FR)

(72) Inventors: Marie-Ange RENAULT, Pessac (FR); Candice CHAPOULY, Pessac (FR)

(21) Appl. No.: 17/995,883

(22) PCT Filed: Apr. 7, 2021
(86) PCT No.: PCT/EP2021/059066

§ 371 Date: Oct. 10, 2022

(30) Foreign Application Priority Data
Apr. 8, 2020 (EP) 20315130.3

(51) Int. Cl. C07K 16/28; A61P 37/06; C12N 15/113

(52) U.S. Cl. CPC C07K 16/2803; A61P 37/06; C12N 15/1138; C12N 2310/11; C12N 2310/14; C12N 2310/12

(57) ABSTRACT

Endothelial dysfunction is a hallmark of peripheral arterial disease which is defined as vascular occlusion below the level of the inguinal ligament, and which is one of the most severe complications of diabetes and inflammatory conditions such as sepsis. Evidences accumulated within the past decades, identified Hedgehog (Hh) signaling as a new regulator of micro-vessel integrity. The purpose of the inventors was to investigate whether Hh co-receptors Gas1 and Cdon may be used as therapeutic targets to modulate Dhh signaling in ECs. The inventors demonstrated that both Gas1 and Cdon are expressed in adult ECs and relied on either siRNAs or EC specific conditional KO mice to investigate their role. They found that Gas1 deficiency mainly photocopies Dhh deficiency especially by inducing VCAM-1 and ICAM-1 overexpression while Cdon deficiency has opposite effects by promoting endothelial junction integrity. At a molecular level, Cdon prevents Dhh binding to Ptch1 and thus acts a decoy receptor for Dhh, while Gas1 promotes Dhh binding to Smo and as a result potentiates Dhh effects. Since Cdon is overexpressed in ECs treated by inflammatory cytokines including TNFα and Il1β, the inventors then tested whether Cdon inhibition would promote endothelium integrity in acute inflammatory conditions and found that both fibrinogen and IgG extravasation were decreased in association with an increased Cdh5 expression in the brain cortex of EC specific Cdon KO mice administered locally with Il1β. Altogether these results demonstrate that Cdon is a negative regulator and justify that Cdon blocking molecules may be used to promote endothelium integrity at least in inflammatory conditions.

### FIELD OF THE INVENTION

The present invention is in the field of medicine, and in particular regenerative medicine.

### BACKGROUND OF THE INVENTION

Endothelium integrity is essential to vascular homeostasis, since a failure of this system represents a critical factor in cardiovascular and cerebrovascular disease pathogenesis. Indeed, the endothelium is involved in many physiological processes such as vascular permeability, vascular tone, blood coagulation and regulation as well as homing of immune cells to specific sites of the body. Conversely, endothelial dysfunction is associated with excessive vasoconstriction especially because of impaired endothelial nitric oxide (NO) production. Also, endothelial dysfunction is characterized by abnormal vascular leakage due to altered endothelial intercellular junctions. Finally, dysfunctional endothelial cells (ECs) acquire pro-inflammatory and pro-thrombotic phenotypes by expressing increased levels of adhesion and pro-thrombotic molecules such as vascular cell adhesion molecule-1 (VCAM-1) and intercellular adhesion molecule-1 (ICAM-1).

Evidences accumulated within the past decades, identified Hedgehog (Hh) signaling as a new regulator of micro-vessel integrity (Candice Chapouly et al. 2019a). For instance, Hh signalling was shown to promote blood brain barrier integrity and immune quiescence both in the setting of multiple sclerosis (Alvarez et al. 2011) and in the setting of stroke (Xia et al. 2013). Additionally we have shown that disruption of Hh signalling specifically in ECs induces blood-nerve barrier breakdown and peripheral nerve inflammation (C. Chapouly et al. 2016). While investigating molecular mechanisms underlying Hh dependent micro-vessel integrity, we found that endothelium “good function” depends on Desert Hedgehog (Dhh) expression by ECs themselves. More specifically, Dhh KO in ECs leads to the disruption of Cadherin-5/beta-catenin interaction and spontaneous vascular leakage and to an increased expression of adhesion molecules including VCAM-1 and ICAM-1 (Caradu et al. 2018). Importantly Dhh which is upregulated by blood flow and downregulated by inflammatory cytokines, appears to be a downstream effector of the master regulator of endothelial integrity Kruppel like factor 2 (Klf2) (Caradu et al. 2018).

The Hedgehog (Hh) family of morphogens which includes Sonic hedgehog (Shh), Indian hedgehog (Ihh) and Dhh, was identified nearly 4 decades ago in drosophila as crucial regulators of cell fate determination during embryogenesis (Nusslein-Volhard et Wieschaus 1980). The interaction of Hh proteins with their specific receptor Patched-1 (Ptch1) de-represses the transmembrane protein Smoothened (Smo), which activates downstream pathways, including the Hh canonical pathway leading to the activation of Gli family zinc finger (Gli) transcription factors and so-called Hh non canonical pathways, which are independent of Smo and/or Gli (Robbins, Fei, et Riobo 2012).

The Hh ligand binding to Ptch1 is regulated by several coreceptors. Among these, Cell adhesion molecule-related/downregulated by oncogenes (Cdon), Brother of Cdon (Boc) and Growth arrest specific 1 (Gas1) are suggested to promote Hh ligand interaction with Ptch1 while Hedgehog interacting protein (Hhip) inhibits it (Ramsbottom et Pownall 2016).

Cdon and Boc proteins are cell surface glycoproteins belonging to a subgroup of the Immunoglobulin superfamily of cell adhesion molecules, which also includes the Robo axon-guidance receptors. Their ectodomain respectively contains five and four Ig-like domains, followed by three FNIII repeats (Fn1-3), a single trans-membrane domain and a divergent intracellular region of variable length (Sanchez-Arrones et al. 2012). Cdon was shown to interact with all of the three N-terminal active Hh peptides (Hh-N) through its Fn3 domain.

Gas1 was identified as one of six genes that were transcriptionally up-regulated in NIH3T3 cells arrested in cell cycle upon serum starvation. Gas1 encodes a 45-kDa GPI-anchored cell surface protein that binds Shh-N with high affinity (Lee, Buttitta, et Fan 2001a).

### SUMMARY OF THE INVENTION

As defined by the claims, the present relates to use of Cdon inhibitors for the treatment of endothelial dysfunction.

### DETAILED DESCRIPTION OF THE INVENTION

Evidences accumulated within the past decades, identified Hedgehog (Hh) signaling as a new regulator of micro-vessel integrity. More specifically, the inventors recently identified Desert Hedgehog (Dhh) as a downstream effector of Klf2 in endothelial cells (ECs). The purpose of this study is to investigate whether Hh co-receptors Gas1 and Cdon may be used as therapeutic targets to modulate Dhh signaling in ECs.

The inventors demonstrated that both Gas1 and Cdon are expressed in adult ECs and relied on either siRNAs or EC specific conditional KO mice to investigate their role. They found that Gas1 deficiency mainly photocopies Dhh deficiency especially by inducing VCAM-1 and ICAM-1 overexpression while Cdon deficiency has opposite effects by promoting endothelial junction integrity. At a molecular level, Cdon prevents Dhh binding to Ptch1 and thus acts a decoy receptor for Dhh, while Gas1 promotes Dhh binding to Smo and as a result potentiates Dhh effects. Since Cdon is overexpressed in ECs treated by inflammatory cytokines including TNFα and Il1β, the inventors then tested whether Cdon inhibition would promote endothelium integrity in acute inflammatory conditions and found that both fibrinogen and IgG extravasation were decreased in association with an increased Cdh5 expression in the brain cortex of EC specific Cdon KO mice administered locally with Il1β.

Altogether these results demonstrate that Gas1 is a positive regulator of Dhh in ECs while Cdon is a negative regulator. Interestingly Cdon blocking molecules may then be used to promote endothelium integrity at least in inflammatory conditions.

Accordingly, the first object of the present invention relates to a method of treating endothelial dysfunction in a patient in need thereof comprising administering to the patient a therapeutically effective amount of a Cdon inhibitor.

As used herein, the term “endothelial dysfunction” has its general meaning in the art and refers to a condition in which the endothelium loses its physiologic properties and shifts toward a vasoconstrictor, prothrombotic, and proinflammatory state. Endothelial dysfunction has been associated with a variety of processes, including hypertension, atherosclerosis, aging, heart and renal failure, coronary syndrome, obesity, vasculitis, infections, sepsis, rheumatoid arthritis, thrombosis, smoking as well as with type 1 and type 2 diabetes.

In some embodiments, endothelial dysfunction is associated with diseases resulting from ischemia and/or reperfusion injury of organs and/or of parts of the body selected from the group comprising heart, brain, peripheral limb, kidney, liver, spleen and lung, and/or wherein the endothelial dysfunction is associated with diseases selected from a group comprising infarctions such as myocardial infarction and critical limb ischemia, and/or wherein the endothelial dysfunction is associated with diseases selected from the group comprising ischemic diseases such as peripheral arterial occlusive disease, e.g. critical leg ischemia, myocardial infarction and ischemic diseases of organs, e.g. of the kidney, spleen, brain and lung.

In some embodiments, the method of the present invention is particularly suitable for the treatment of endothelial dysfunction in a patient suffering from a systemic inflammatory response syndrome or sepsis. As used herein, the term “systemic inflammatory response syndrome” (or “SIRS”) is in accordance with its normal meaning, to refer to an inflammatory state of the whole body without a source of infection. There are four major diagnostic symptoms of SIRS, although any two of these are enough for a diagnosis (see e.g. Nystrom (1998) Journal of Antimicrobial Chemotherapy, 41, Suppl A, 1-7). As used herein, the term “sepsis” refers to a form of SIRS which is caused by a suspected or proven infection (see e.g. Nystrom (1998) Journal of Antimicrobial Chemotherapy, 41, Suppl. A, 1-7). An infection that leads to sepsis may be caused by e.g. a virus, a fungus, a protozoan or a bacterium.

In some embodiments, the method of the present invention is particularly suitable for the treatment of endothelial dysfunction in a patient suffering from diabetes mellitus. As used herein, the term “diabetes mellitus” refers to a disease caused by a relative or absolute lack of insulin leading to uncontrolled carbohydrate metabolism, commonly simplified to “diabetes,” though diabetes mellitus should not be confused with diabetes insipidus. As used herein, “diabetes” refers to diabetes mellitus, unless otherwise indicated. A “diabetic condition” includes pre-diabetes and diabetes. Type 1 diabetes (sometimes referred to as “insulin-dependent diabetes” or “juvenile-onset diabetes”) is an auto-immune disease characterized by destruction of the pancreatic β cells that leads to a total or near total lack of insulin. In type 2 diabetes (T2DM; sometimes referred to as “non-insulin-dependent diabetes” or “adult-onset diabetes”), the body does not respond to insulin, though it is present.

Accordingly, in some embodiments, the present invention is particularly suitable for the treatment of diabetic micro- and/or macroangiopathy. In some embodiments, the method of the present invention is particularly suitable for the treatment of diabetic nephropathy, diabetic dermopathy, diabetic retinopathy and diabetic neuropathy.

In some embodiments, the method of the present invention is particularly suitable for the treatment of a peripheral arterial disease. As used herein, the term “peripheral arterial disease” or “PAD” refers to acute and chronic critical limb ischemia, Buerger's disease and critical limb ischemia in diabetes. As used herein, the term “critical limb ischemia” or “CLI” generally refers to a condition characterized by restriction in blood or oxygen supply to the extremities (e.g., hands, feet, legs) of an individual that may result in damage or dysfunction of a tissue in the extremities. Critical limb ischemia may cause severe pain, skin ulcers, or sores, among other symptoms, and in some cases leads to amputation. Critical limb ischemia may be characterized by vasoconstriction, thrombosis, or embolism in one or more extremities. Any tissue in an extremity that normally receives a blood supply can experience critical limb ischemia. In particular, the Hh agonist of the present invention is particularly suitable for promoting muscle perfusion and for preventing myopathy in the setting of critical limb ischemia.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “Cdon” has its general meaning in the art and refers to the protein named “cell adhesion molecule-related/down-regulated by oncogenes”. Typically, Cdon has an amino acid sequence as set forth in SEQ ID NO:1. The extracellular domain of Cdon ranges from the amino acid at position 26 to the amino acid at position 963 in SEQ ID NO:1.

>sp|Q4KMG0|CDON_HUMAN Cell adhesion molecule-
related/down-regulated by oncogenes OS = Homo
sapiens OX = 9606 GN = CDON PE = 1 SV = 2
SEQ ID NO: 1
MHPDLGPLCTLLYVTLTILCSSVSSDLAPYFTSEPLSAVQKLGGPVVLH
CSAQPVTTRISWLHNGKTLDGNLEHVKIHQGTLTILSLNSSLLGYYQCL
ANNSIGAIVSGPATVSVAVLGDFGSSTKHVITAEEKSAGFIGCRVPESN
PKAEVRYKIRGKWLEHSTENYLILPSGNLQILNVSLEDKGSYKCAAYNP
VTHQLKVEPIGRKLLVSRPSSDDVHILHPTHSQALAVLSRSPVTLECVV
SGVPAPQVYWLKDGQDIAPGSNWRRLYSHLATDSVDPADSGNYSCMAGN
KSGDVKYVTYMVNVLEHASISKGLQDQIVSLGATVHFTCDVHGNPAPNC
TWFHNAQPIHPSARHLTAGNGLKISGVTVEDVGMYQCVADNGIGFMHST
GRLEIENDGGFKPVIITAPVSAKVADGDFVTLSCNASGLPVPVIRWYDS
HGLITSHPSQVLRSKSRKSQLSRPEGLNLEPVYFVLSQAGASSLHIQAV
TQEHAGKYICEAANEHGTTQAEASLMVVPFETNTKAETVTLPDAAQNDD
RSKRDGSETGLLSSFPVKVHPSAVESAPEKNASGISVPDAPIILSPPQT
HTPDTYNLVWRAGKDGGLPINAYFVKYRKLDDGVGMLGSWHTVRVPGSE
NELHLAELEPSSLYEVLMVARSAAGEGQPAMLTFRTSKEKTASSKNTQA
SSPPVGIPKYPVVSEAANNNFGVVLTDSSRHSGVPEAPDRPTISTASET
SVYVTWIPRANGGSPITAFKVEYKRMRTSNWLVAAEDIPPSKLSVEVRS
LEPGSTYKFRVIAINHYGESFRSSASRPYQVVGFPNRFSSRPITGPHIA
YTEAVSDTQIMLKWTYIPSSNNNTPIQGFYIYYRPTDSDNDSDYKRDVV
EGSKQWHMIGHLQPETSYDIKMQCFNEGGESEFSNVMICETKVKRVPGA
SEYPVKDLSTPPNSLGSGGNVGPATSPARSSDMLYLIVGCVLGVMVLIL
MVFIAMCLWKNRQQNTIQKYDPPGYLYQGSDMNGQMVDYTTLSGASQIN
GNVHGGFLTNGGLSSGYSHLHHKVPNAVNGIVNGSLNGGLYSGHSNSLT
RTHVDFEHPHHLVNGGGMYTAVPQIDPLECVNCRNCRNNNRCFTKTNST
FSSSPPPVVPVVAPYPQDGLEMKPLSHVKVPVCLTSAVPDCGQLPEESV
KDNVEPVPTQRTCCQDIVNDVSSDGSEDPAEFSRGQEGMINLRIPDHLQ
LAKSCVWEGDSCAHSETEINIVSWNALILPPVPEGCAEKTMWSPPGIPL
DSPTEVLQQPRET

As used herein, the term “Cdon inhibitor” refers to a compound, substance or composition that can inhibit the function and/or expression of Cdon. For example, the inhibitor can inhibit the expression or activity of Cdon, modulate or block the Cdon or block the signalling pathway. In particular, the inhibitor of Cdon inhibits the interaction between Cdon and its partners, in particular Desert Hedgehog (Dhh).

As used herein, the term “Desert Hedgehog” or “Dhh” has its general meaning in the art and refers to the desert hedgehog protein encode by the DHH gene (Gene ID: 50846). Typically, Dhh has an amino acid sequence as set forth in SEQ ID NO:2. The N-terminal domain ranges from the amino acid at position 23 to the amino acid at position 198 in SEQ ID NO:2.

>sp|O43323|DHH_HUMAN Desert hedgehog protein OS =
Homo sapiens OX = 9606 GN = DHH PE = 1 SV = 1
SEQ ID NO: 2
MALLTNLLPLCCLALLALPAQSCGPGRGPVGRRRYARKQLVPLLYKQFV
PGVPERTLGASGPAEGRVARGSERFRDLVPNYNPDIIFKDEENSGADRL
MTERCKERVNALAIAVMNMWPGVRLRVTEGWDEDGHHAQDSLHYEGRAL
DITTSDRDRNKYGLLARLAVEAGFDWVYYESRNHVHVSVKADNSLAVRA
GGCFPGNATVRLWSGERKGLRELHRGDWVLAADASGRVVPTPVLLFLDR
DLQRRASFVAVETEWPPRKLLLTPWHLVFAARGPAPAPGDFAPVFARRL
RAGDSVLAPGGDALRPARVARVAREEAVGVFAPLTAHGTLLVNDVLASC
YAVLESHQWAHRAFAPLRLLHALGALLPGGAVQPTGMHWYSRLLYRLAE
ELLG

In some embodiments, the Cdon inhibitor is an antibody having binding affinity for Cdon. In some embodiments, the Cdon inhibitor is an antibody directed against the extracellular domain of Cdon. In some embodiments, the antibody of the present invention is capable of inhibiting the binding of Cdon to Dhh. In some embodiments, the Cdon inhibitor is an antibody having binding affinity for the region of Cdon which binds to Dhh. In some embodiments, the antibody binds to Fibronectin type-III 3 domain of Cdon. In some embodiments, the Cdon inhibitor is an antibody having binding affinity for the amino acid sequence ranging from the amino acid residue at position 826 to the amino acid residue at position 926 in SEQ ID NO:1.

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments.

In some embodiments, the antibody is a humanized antibody. Methods of humanization include, but are not limited to, those described in U.S. Pat. Nos. 4,816,567, 5,225,539, 5,585,089, 5,693,761, 5,693,762 and 5,859,205, which are hereby incorporated by reference.

In some embodiments, the antibody is a fully human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference.

In some embodiments, the antibody of the present invention is a single chain antibody.

In some embodiments, the antibody comprises human heavy chain constant regions sequences but will not induce antibody dependent cellular cytotoxicity (ADCC). In some embodiments, the antibody of the present invention does not comprise an Fc domain capable of substantially binding to a FcgRIIIA (CD16) polypeptide. In some embodiments, the antibody of the present invention lacks an Fc domain (e.g. lacks a CH2 and/or CH3 domain) or comprises an Fc domain of IgG2 or IgG4 isotype. In some embodiments, the antibody of the present invention consists of or comprises a Fab, Fab′, Fab′-SH, F (ab′) 2, Fv, a diabody, single-chain antibody fragment, or a multispecific antibody comprising multiple different antibody fragments. In some embodiments, the antibody of the present invention is not linked to a toxic moiety. In some embodiments, one or more amino acids selected from amino acid residues can be replaced with a different amino acid residue such that the antibody has altered C2q binding and/or reduced or abolished complement dependent cytotoxicity (CDC). This approach is described in further detail in U.S. Pat. No. 6,194,551 by ldusogie et al.

In some embodiments, the Cdon inhibitor is an inhibitor of Cdon expression.

An “inhibitor of expression” refers to a natural or synthetic compound that has a biological effect to inhibit the expression of a gene.

In some embodiments, said inhibitor of gene expression is a siRNA, an antisense oligonucleotide or a ribozyme. For example, anti-sense oligonucleotides, including anti-sense RNA molecules and anti-sense DNA molecules, would act to directly block the translation of Cdon mRNA by binding thereto and thus preventing protein translation or increasing mRNA degradation, thus decreasing the level of Cdon, and thus activity, in a cell. For example, antisense oligonucleotides of at least about 15 bases and complementary to unique regions of the mRNA transcript sequence encoding Cdon can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. Cdon gene expression can be reduced by contacting a patient or cell with a small double stranded RNA (dsRNA), or a vector or construct causing the production of a small double stranded RNA, such that Cdon gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing Cdon. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In some embodiments, the inhibitor of expression is an endonuclease. In a particular embodiment, the endonuclease is CRISPR-cas. In some embodiment, the endonuclease is CRISPR-cas9 which is from Streptococcus pyogenes. The CRISPR/Cas9 system has been described in U.S. Pat. No. 8,697,359 B1 and US 2014/0068797. In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

By a “therapeutically effective amount” of the inhibitor of the invention as above described is meant a sufficient amount of the inhibitor according to the present invention. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The inhibitor of the invention may be combined with pharmaceutically acceptable excipients, and optionally sustained-release matrices, such as biodegradable polymers, to form therapeutic compositions. “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Galenic adaptations may be done for specific delivery in the small intestine or colon. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising inhibitors of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The inhibitor of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifusoluble agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. The inhibitor of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered. In addition to the inhibitors of the invention formulated for parenteral administration, such as intravenous or intramuscular injection, other pharmaceutically acceptable forms include, e.g. tablets or other solids for oral administration; liposomal formulations; time release capsules; and any other form currently used.

The invention will be further illustrated by the following figures and examples. However, these examples and figures should not be interpreted in any way as limiting the scope of the present invention.

### FIGURES

FIG. 1: Cdon deficiency in ECs prevents Il1β-induced BBB disruption. Both Cdh5-Cre^{ERT2}; Cdon^{Flox/Flox} (Cdon^{ECKO}) and Cdon^{Flox/Flox} (control) mice were administered in the cerebral cortex with adenoviruses encoding Il1β (n=7 and 5 mice respectively). Mice were sacrificed 7 days later. (A) Cdh5 expression was quantified as the Cdh5+ surface area. (B) Fibrinogen extravasation was quantified as the fibrinogen+ surface area. (C) IgG extravasation was quantified as the fibrinogen+ surface area. *: p≤0.05; **: p≤0.01; ***: p≤0.001. Mann Withney test.

FIG. 2: Cdon blocking antibodies can be used to promote endothelium integrity. HUVECs were treated or not with 10 ng/mL TNFα in the presence of 1.5 μg/mL Cdon blocking antibodies or 1.5 μg/mL unspecific IgGs. Cdh5 localization was quantified as the mean junction thickness using Image J software. The experiment was repeated at least 4 times. **: p≤0.01; ***: p≤0.001. NS: not significant. One way ANOVA followed by Bonferroni's multiple comparisons test.

### EXAMPLE

Material & Methods

Mice

Cdon Floxed (Cdon^{Flox}) mice were generated at the “Institut Clinique de la Souris” through the International Mouse Phenotyping Consortium (IMPC) from a vector generated by the European conditional mice mutagenesis program, EUCOMM. Gas1^{tm3,1Fan} (Gas1^{Flox}) mice (Jin et al. 2015, 1) were kindly given by C. M. Fan and Tg(Cdh5-cre/ERT2)1Rha (Cdh5-CreERT2) mice (Azzoni et al. 2014) were a gift from RH. Adams.

Animal experiments were performed in accordance with the guidelines from Directive 2010/63/EU of the European Parliament on the protection of animals used for scientific purposes and approved by the local Animal Care and Use Committee of Bordeaux University.

The Cre recombinase in Cdh5-Cre^{ERT2} mice was activated by intraperitoneal injection of 1 mg tamoxifen for 5 consecutive days at 8 weeks of age. Mice were phenotyped 2 weeks later. Successful and specific activation of the Cre recombinase has been verified before (Caradu et al. 2018). Both males and females were used in equal proportions. At the end of experiments animal were sacrificed via cervical dislocation.

Mouse Corneal Angiogenesis Assay

Pellets were prepared as previously described (Kenyon et al. 1996). Briefly, 5 μg of VEGFA (Shenandoah biotechnology diluted in 10 μL sterile phosphate-buffered saline (PBS) was mixed with 2.5 mg sucrose octasulfate-aluminum complex (Sigma-Aldrich Co., St. Louis, Mo., USA), and 10 μL of 12% hydron in ethanol was added. The suspension was deposited on a 400-μm nylon mesh (Sefar America Inc., Depew, N.Y., USA), then both sides of the mesh were covered with a thin layer of hydron and allowed to dry.

Female mice were anesthetized with an intraperitoneal (IP) injection of ketamine 100 mg/kg and xylazine 10 mg/kg. The eyes of the mice eyes were topically anesthetized with 0.5% Proparacaine™ or similar ophthalmic anesthetic. The globe of the eye was proptosed with jeweler's forceps taking care to not damage the limbus vessel surrounding the base of the globe. Sterile saline was also be applied directly to each eye as needed during the procedure to prevent excessive drying of the cornea and to facilitate insertion of the pellet into the lamellar pocket of the eyes. Using an operating microscope, a central, intrasomal linear keratotomy was performed with a surgical blade parallel to the insertion of the lateral rectus muscle. Using a modified von greafe knife, a lamellar micro pocket was made toward the temporal limbus by ‘rocking’ the von greafe knife back and forth.

Hh containing or control pellet was placed on the cornea surface with jeweler's forceps at the opening of the lamellar pocket. A drop of saline was applied directly to the pellet, and using the modified von greafe knife, the pellet was gently advanced to the temporal end of the pocket. Buprenorphine was given at a dose of 0.05 mg/kg subcutaneously on the day of surgery.

Nine days after pellet implantation, mice were sacrificed, and then eyes were harvested and fixed with 2% paraformaldehyde. Capillaries were stained with rat anti-mouse CD31 antibodies (BMA Biomedicals, Cat #T-2001), primary antibodies were visualized with Alexa 568-conjugated anti-rat antibodies (Invitrogen). Pictures were taken under 50× magnification. Angiogenesis was quantified as the CD31+surface area.

In Vivo Permeability Assessment (Miles Assay)

The back of female mice was shaved. 72 hours, later mice were administered with 100 μL 1% Evans blue via retro orbital injection. Subsequently they were administered with 50 μL NaCl 0.9% containing or not 20 ng VEGFA (Shenandoah biotechnology) subcutaneously at 6 spots on their back.

30 minutes later mice were sacrificed, skin biopsy around each injection point were then harvested to quantify Evans blue extravasation. Evans blue dye was extracted from the skin by incubation at 65° C. with formamide. The concentration of Evans blue dye extracted was determined spectrophotometrically at 620 nm with a reference at 740 nm. Buprenorphine was given at a dose of 0.05 mg/kg subcutaneously on the day of surgery.

Ad-Il1β Stereotaxic Injections

Mice were anaesthetized using isoflurane and placed into a stereotactic frame (Stoelting). To prevent eye dryness, an ophthalmic ointment was applied at the ocular surface to maintain eye hydration during the time of surgery. The skull was shaved and the skin incised on 1 cm to expose the skull cap. Then, a hole was drilled into the cerebral cortex and 3 μL of an AdIL-1 (Horng et al. 2017) or AdDL70 control (AdCtrl), (10⁷ pfu) solution was microinjected at y=1 mm caudal to Bregma, x=2 mm, z=1.5 mm using a Hamilton syringe, into the cerebral cortex and infused for 3 minutes before removing the needle from the skull hole (Argaw et al. 2009). Mice received a subcutaneous injection of buprenorphine (0.05 mg/kg) 30 minutes before surgery and again 8 hours post-surgery to assure a constant analgesia during the procedure and postoperatively. Mice were sacrificed 7 days post-surgery. For histological assessment, brains were harvested and fixed in formalin for 3 hours before being incubated in 30% sucrose overnight and OCT embedded. Then, for each brain, the lesion area identified by the puncture site was cut into 7 μm thick sections.

Immunostaining

Prior to staining, heart, brain, and lung tissues were fixed in methanol; paraffin embedded and cut into 7 μm thick sections. Whole mount corneas were fixed with 2.5% Formaline for 10 minutes and cultured cells were fixed with 10% formaline for 10 minutes.

Capillaries were identified using rat anti-mouse CD31 antibodies (BMA Biomedicals, Cat #T-2001). Neutrophils were stained with a rat anti-Ly6G (GR1) antibody (BD Pharmingen Inc, Cat #551459). Human Cdh5 was stained using mouse anti-human Cdh5 antibodies (Santa Cruz Biotechnology, Inc, Cat #sc-9989). Mouse Cdh5 was stained using goat anti-mouse Cdh5 antibodies (R&D systems, Cat #AF1002). Albumin and fibrinogen were stained using sheep anti-albumin antibodies (Abcam, Cat #ab8940) and rabbit anti-fibrinogen antibodies (Dako, Cat #A0080) respectively. Mouse IgGs were stained with Alexa Fluor 568 conjugated donkey anti-mouse IgG (Invitrogen, Cat #A-10037). Pan-leucocytes were identified using rat anti-mouse CD45 antibodies (BD Pharmingen Inc, Cat #550539). CD11b+ microglia and macrophages were identified using rat anti-CD11b antibodies (ThermoFisher, cat #14-0112-82). GFAP was stained using rabbit anti-GFAP antibodies (ThermoFisher, Cat #OPA1-06100). Neurons were identified using anti-NeuN antibodies (Millipore, Cat #ABN78). Cdon was stained using goat anti-mouse Cdon antibodies (R&D systems, Cat #AF2429). Gas1 was stained using goat anti-human Gas1 antibodies (R&D systems, Cat #AF2636). Dhh was stained using mouse anti-Dhh antibodies (Santa Cruz Biotechnology, Inc, Cat #sc-271168). Ptch1 was stained using rabbit anti-Ptch1 antibodies (Abcam, Cat #ab53715). For immunofluorescence analyzes, primary antibodies were resolved with Alexa Fluor®-conjugated secondary polyclonal antibodies (Invitrogen, Cat #A-21206, A-21208, A-11077, A-11057, A-31573, A-10037) and nuclei were counterstained with DAPI (1/5000). For both immunohistochemical and immunofluorescence analyses, negative controls using secondary antibodies only were done to check for antibody specificity.

Cell Culture

In vitro experiments were performed using human umbilical vein endothelial cells (HUVECs) (Lonza), human dermal microvascular endothelial cells (HMVECs-D) (Lonza) or human brain microvascular Endothelial Cells (HBMECs) (Alphabioregen). HUVECs and HBMECs were cultured in endothelial basal medium-2 (EBM-2) supplemented with EGM™-2 BulletKits™ (Lonza). HMVECs-D were cultured in endothelial basal medium-2 (EBM-2) supplemented with EGM™-2 MV BulletKits™ (Lonza). Cell from passage 3 to passage 6 were used. Before any treatment cells were serum starved in 0.5% fetal bovine serum medium for 24 hours. HeLa ATCC®CCL-2™ cells were cultured in Roswell Park Memorial Institute medium (RPMI) supplemented with 10% fetal bovine serum.

siRNA/Transfection

HUVECs were transfected with human Gas1 siRNA, human Cdon siRNA, human Dhh siRN, human Ptch1 siRNA or universal scrambled negative control siRNA duplex (Origen) using JetPRIME™ transfection reagent (Polyplus Transfection), according to the manufacturer's instructions.

Plasmids/Transfection

The human Gas1 encoding vector, pcDNA3-Gas1 was kindly given by C. M. Fan (Lee, Buttitta, et Fan 2001a), the GFP tagged-mouse Cdon encoding vector, pCA-mCdonEGFP, was a gift from A. Okada (Okada et al. 2006), the myc-tagged human Ptch1, Ptch1-1B-myc was kindly given by R. Toftgard (Kogerman et al. 2002) and the human full length Dhh was previously described (Caradu et al. 2018). A myc tag was added by PCR at the N-terminal of human full length Dhh to generate the myc-tagged Dhh encoding vector.

HeLa cells were transfected using JetPRIME™ transfection reagent (Polyplus Transfection), according to the manufacturer's instructions.

Quantitative Reverse-Transcription Polymerase Chain Reaction (RT-PCR)

Following manufacturer's instructions, RNAs were isolated and homogenized, from 3×10⁵ cells or from tissues previously snap-frozen in liquid nitrogen, using Tri Reagent® (Molecular Research Center Inc). For quantitative RT-PCR analyzes, total RNA was reverse transcribed with M-MLV reverse transcriptase (Promega) and amplification was performed on a DNA Engine Opticon®2 (MJ Research Inc) using B-R SYBER® Green SuperMix (Quanta Biosciences). The relative expression of each mRNA was calculated by the comparative threshold cycle method and normalized to β-actin mRNA expression.

Immunoprecipitation/Western Blot Analysis

Prior to western blot analysis, Dhh, Ptch1 ou Smo were immunoprecipitated with mouse anti-Dhh antibodies (Santa Cruz Biotechnology, Cat #sc-271168), anti myc-tag antibodies (Millipore, Cat #05-724) or mouse anti-Smo antibodies (Santa Cruz Biotechnology, Cat #sc-166685).

Expression of Cdon, Gas1, Dhh, Ptch1 and Smo were evaluated by SDS PAGE using goat anti-mouse Cdon antibodies (R&D systems, Cat #AF2429), goat anti-human Gas1 antibodies (R&D systems, Cat #AF2636), mouse anti-Dhh antibodies (Santa Cruz Biotechnology, Inc, Cat #sc-271168), rabbit anti-Ptch1 antibodies (Abcam, Cat #ab53715) and mouse anti-Smo antibodies (Santa Cruz Biotechnology, Cat #sc-166685) respectively.

Expression of human ICAM-1 and VCAM-1 expression were evaluated by SDS PAGE using mouse anti-human I-CAM1 antibodies (Santa Cruz Biotechnology, Cat #sc-8439) and rabbit anti-VCAM-1 (Abcam, Cat #ab134047) respectively.

Protein loading quantity was controlled using a monoclonal anti-α-tubulin antibody (Sigma). Secondary antibodies were from Invitrogen, Cat #A-21039, A-21084, A-21036). The signal was then revealed by using an Odyssey Infrared imager (LI-COR).

In Vitro Permeability Assay

100 000 cells were seeded in Transwell® inserts. The day after, 0.5 mg/mL 70 kDa FITC-Dextran (Sigma) was added to the upper chamber. FITC fluorescence in the lower chamber was measured 20 minutes later.

Migration Assay

Cell migration was evaluated with a chemotaxis chamber (Neuro Probe, Inc., Gaithersburg, Md., USA). Briefly, a polycarbonate filter (8-μm pore size) (GE Infrastructure, Fairfield, Conn., USA) was coated with a solution containing 0.2% gelatin (Sigma-Aldrich Co.) and inserted between the chambers, then 5×104 cells per well were seeded in the upper chamber, and the lower chamber was filled with EBM-2 medium containing 0.5% FBS. Cells were incubated for 8 hours at 37° C. then viewed under 20× magnification, and the number of cells that had migrated to the lower chamber were counted in 3 HPFs per well; migration was reported as the mean number of migrated cells per HPF. Each condition was assayed in triplicate and each experiment was performed at least three times.

Methyl Thiazolyl Tetrazolium (MTT) Cell Proliferation Assay

5×10³ cells per well were seeded in a 96-well plate. At the indicated time points, 10 of 5 mg/mL MTT were added to each wells. Cells were incubated 3-4 hours at 37° C. then culture medium was replaced by 100 μL DMSO. OD was read at 590 nm with a reference at 620 nm. Each condition included eight wells in each experiment and each experiment was performed at least three times.

Statistics

Results are reported as mean±SEM. Comparisons between groups were analyzed for significance with the non-parametric Mann-Whitney test or a one way ANOVA test followed by Bonferroni's multiple comparison test (for than two groups) using GraphPad Prism v7.0 (GraphPad Inc, San Diego, Calif.). Differences between groups were considered significant when p<0.05 (*: p<0.05; **: p<0.01; ***: p<0.001).

Results

ECs Express Cdon, Gas1 and Hhip but not Boc.

First, we searched for Cdon, Boc, Gas1 and Hhip expression in human EC from different origin, including HUVECs, HMVECs-D and HBMECs via RT-PCR. We show that human ECs express Hhip, Cdon, and Gas1 while they barely express Boc (data not shown). Notably, Gas1 is not detected in HBMECs. Since the role of endothelial Hhip has already been reported in several papers (Agrawal, Kim, et Kwon 2017; Sekiguchi et al. 2012; Nie et al. 2016), we focused our investigations on Gas1 and Cdon and confirmed their endothelial expression via immunostaining (data not shown). Interestingly, while TNFα inhibits Gas1 mRNA expression in HUVECs (data not shown), it increases Cdon mRNA expression (data not shown). Moreover, TNFα-induced Cdon mRNA expression depends on NF-κB activity (data not shown).

Next, we performed co-immunoprecipitation assays to verify that Dhh is able to bind these receptors. While we found that Dhh binds Gas1 directly (data not shown), Cdon alone cannot bind Dhh (data not shown). However, Dhh can bind Cdon in the presence of Ptch1 (data not shown). Consistently, Cdon co-localizes with Dhh only in the presence of Ptch1 while Gas1 co-localizes with Dhh both in the presence and absence of Ptch1 (data not shown)

With the aim to further investigate the role of Gas1 and Cdon in ECs we performed a series of in vitro and in vivo assays using siRNAs and EC-specific conditional KO mice respectively.

Cdon Promotes EC Proliferation, Migration and Angiogenesis

The role of Gas1 and Cdon in angiogenesis was investigated using the mouse corneal angiogenesis assay. Mice deficient for Gas1 or Cdon expression in EC together with their respective control littermates were implanted with VEGFA containing pellets. While VEGFA-induced angiogenesis was not different in Gas1^{ECKO} mice from their control littermates (data not shown), VEGFA-induced angiogenesis was significantly-inhibited in Cdon^{ECKO} mice compared to their control littermates (data not shown). Consistently, in vitro experiments performed in HUVECs showed that both EC proliferation (data not shown) and VEGFA-induced EC migration (data not shown) were decreased after Cdon knock down (KD). Gas1 KD did not modify EC proliferation or VEGFA-induced migration. However, Gas1 KD did promote EC migration in the absence of VEGF (data not shown).

This set of data demonstrates that Cdon is pro-angiogenic. Notably this effect works in the opposite direction to Dhh anti-angiogenic effect (Hollier et al. 2020).

Gas1 Prevent EC Activation and LPS-Induced Neutrophil Recruitment.

To investigate the role of Gas1 and Cdon in regulating EC immune quiescence, HUVECs were transfected with Gas1, Cdon or control siRNAs. VCAM-1 and ICAM-1 expression was measured via both RT-qPCR and western blot analyses. While Gas1 KD significantly increased both VCAM-1 and ICAM-1 expression (data not shown), Cdon KD did not (data not shown). These results were confirmed in TNFα treated cells (data not shown). Finally, to assess the functional consequences of EC activation in vivo, we quantified neutrophils recruitment in the lungs of mice that were administered with LPS. As expected, Neutrophils density in the lung of Gas1^{ECKO} mice was significantly increased (data not shown) compared to their control littermates while we found no difference between Cdon^{ECKO} mice and control littermates (data not shown).

Altogether these data demonstrate that Gas1 prevents EC activation similarly to Dhh (Caradu et al. 2018). On the contrary, Cdon does not seem to participate in the regulation of EC activation.

Cdon Disrupts Adherens Junction Integrity.

The role of Gas1 and Cdon in controlling endothelial intercellular junction integrity was first investigated in vitro. HUVECs were transfected with Gas1, Cdon or control siRNAs. Adherens junction integrity was quantified after Cdh5 immunostaining (data not shown) and endothelium permeability using Transwells. We found that Gas1 KD disrupts Cdh5-dependent junction integrity (data not shown) while Cdon KD prevents EC permeability (data not shown). Gas1 KD did not show any effects in the permeability assay test suggesting a very mild effect of Gas1 on adherens junction integrity. Consistently, in the Miles assay in vivo, VEGFA-induced vascular permeability was not different between Gas1^{ECKO} and control mice (data not shown) while it was significantly decreased in the absence of endothelial Cdon (data not shown).

This last set of data demonstrate that Cdon strongly increases vascular permeability unlike Dhh (Hollier et al. 2020; Caradu et al. 2018) and that Gas 1 may slightly modify Cdh5 junction organization without functional consequences.

To conclude on this first set of results, Gas1 may promote Hh signaling in ECs since Gas1 KD mostly recapitulates the effects of Dhh KD. On the contrary, Cdon most likely inhibits Hh signaling since Cdon KD induces opposite effect to those of Dhh KD. Notably, Cdon has been previously identified as a Hh decoy receptor in the zebrafish optic vesicle (Cardozo et al. 2014).

Therefore, in the second part of this study, we chose to perform a series of experiments aiming to investigate whether and how Gas1 and Cdon modulate Hh signaling in ECs.

Gas1 Promotes Dhh Interaction with Smo while Cdon Prevents Dhh Interaction with Ptch1.

First we tested whether Gas1 or Cdon modulate Dhh interaction with Ptch1 and Smo. Notably, Smo has been recently shown to be a receptor for Hh ligands especially in the case of cell autonomous signaling (Casillas et Roelink 2018). Interestingly, we found that Gas1 prevents Dhh interaction with Ptch1 but promotes Dhh interaction with Smo. On the contrary, Cdon prevents Dhh interaction with Ptch1 but does not modify Dhh interaction with Smo (data not shown).

Since Gas1 KD phenocopies most features of Dhh deficiency, we tested whether Gas1 effects on endothelial adherens junction's integrity and migration depend on Hh signaling. To do so, we performed rescue experiments. HUVECs were either transfected with Gas1 or control siRNAs and then treated or not with the Smo agonist SAG. In particular, we show that Gas1 KD-induced Cdh5 junction thickening was prevented in the presence of SAG (data not shown). Similarly, Gas1 KD failed to induce EC migration in the presence of SAG (data not shown). However, SAG had no effect on Gas1 KD-induced VCAM-1 and ICAM-1 (data not shown). Notably, VCAM-1 and ICAM-1 seem to be downstream of Ptch1 rather than Smo since Ptch1 KD is sufficient to increase their expression (data not shown).

Because Cdon has opposite effects to Dhh ones, we hypothesized that Cdon is a decoy receptor for Dhh at the surface of EC and thus tested whether siCdon-induced effects are prevented in the absence of Dhh. HUVECs were transfected with Cdon siRNAs alone or in combination with Dhh siRNAs. While siCdon alone decreased adherent junction thickness and endothelium permeability, in the siCdon+siDhh condition (data not shown), effects were no longer significant confirming our hypothesis.

Cdon Deficiency at the Endothelium Prevents Blood-Brain Barrier Opening in the Setting of Acute Inflammation

Finally, since Cdon appears to act as a negative regulator of Dhh-induced signaling in ECs, we hypothesized that blocking Cdon may promote Dhh-induced signaling in ECs and subsequently promote maintenance of endothelium integrity in pathological conditions.

To test such hypothesis, we administered adenoviruses encoding Il1β locally in the cortex of both Cdon^{ECKO} mice and their control littermate to induce acute brain inflammation and BBB breakdown.

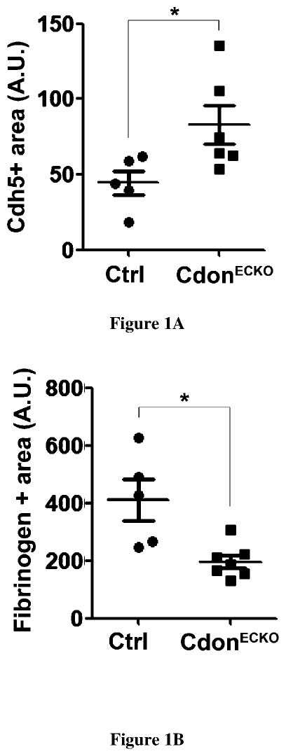

Notably, Cdon expression is significantly increased upon Il1β treatment in both HUVECs and HBMECs (data not shown). In accordance with our hypothesis, endothelial adherens junctions were preserved in the absence of Cdon, as attested by an increased Cdh5 expression in the cortical lesion area of Cdon^{ECKO} mice injected with Il1β, compared to control littermates (FIGS. 1A-B). Consistently, both fibrinogen and IgG extravasation were decreased (FIGS. 1A, C). BBB tightness in Cdon^{ECKO} mice was associated with a decreased leucocyte infiltration, a decreased microglia and astrocyte activation and finally with an increased neuron survival (data not shown).

These last results demonstrate that blocking Cdon might indeed be a working therapeutic strategy to preserve endothelium integrity in pathological setting such as acute neuro-inflammation.

Cdon Blocking Antibodies May be Used as a Therapeutic Tool to Maintain Endothelial Junctions in the Setting of Inflammation

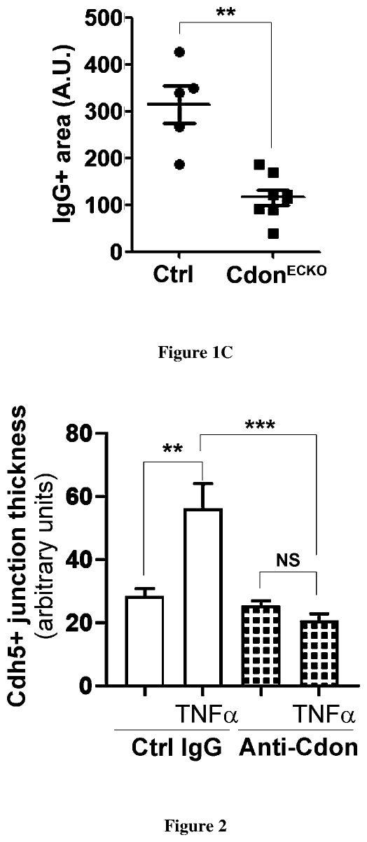

We then tested whether Cdon antibodies may be used as a therapeutic tool to block Dhh binding to Cdon and improve endothelial integrity. To do so, HUVECs were treated or not with TNFα, in the presence or not of Cdon blocking antibodies. As shown in FIG. 2, TNFα-induced Cdh5 junction thickening is prevented in the presence of Cdon blocking antibodies.

### DISCUSSION

Hedgehog signaling has been described to be regulated by several co-receptors including Hhip, Boc, Cdon and Gas1 especially in the setting of embryogenesis (Allen et al. 2011). The purpose of the present study was to investigate the role of Gas1 and Cdon in ECs in adults. Importantly, Hh signaling in ECs is original by several aspects. First, it exclusively involves non canonical signaling (Renault et al. 2010; Chinchilla et al. 2010), second, it is activated by full length unprocessed Dhh (FL-Dhh) (Hollier et al. 2020) and third, it occurs cell autonomously (Caradu et al. 2018). It is important to have in mind that full length unprocessed Hh ligands, may not only bind Ptch1 but also Smo directly (Casillas et Roelink 2018). In this particular setting, the present study demonstrates that Cdon prevents FL-Dhh binding to Ptch1. Gas1 also prevents FL-Dhh binding to Ptch1 but promotes FL-Dhh binding to Smo. By doing so, Cdon mainly acts as a negative regulator of FL-Dhh and destabilizes endothelial cell junctions to promote angiogenesis while Gas 1 is a positive regulator of FL-Dhh which prevents EC activation.

Cdon, Gas1 and Boc are typically believed to be positive regulators of Hh signaling (Ramsbottom et Pownall 2016) in line with the fact that Gas1, Cdon and Boc were shown to be equally capable of promoting Shh signaling during neural patterning since overexpression of any individual component results in ectopic ventral cell fate specification (Allen et al. 2011). Additionally, while genetic removal of Gas1, Cdon or Boc individually has only modest effects on Shh signaling, removal of any two components results in significantly reduced Shh-dependent ventral neural patterning (Allen et al. 2011). However, conflicting results have been published: Gas1 was first shown to bind Shh in 2001. However, it was first suggested to reduce the availability of active Shh in the somite based on ectopic expression studies (Lee, Buttitta, et Fan 2001b). In 2007, experiments using Gas1 deficient mice revealed, on the contrary, that Gas1 is a positive regulator of Shh signaling and facilitates Shh low level effects (Martinelli et Fan 2007). Similarly, Cdon was shown to positively regulate Shh-induced signaling especially in the developing brain (Tenzen et al. 2006; Zhang et al. 2006) while it was more recently shown to act as a Hedgehog decoy receptor during proximal-distal patterning of the optic vesicle (Cardozo et al. 2014). Whether Gas1 and Cdon are positive or negative regulators of Hh signaling may then most likely depend on the type of ligand and cell type involved.

Hedgehog signaling in ECs is still far from being fully understood (Candice Chapouly et al. 2019b). We have previously shown that Dhh prevents EC activation by downregulating VCAM-1 and ICAM-1 and protects adherens junction integrity by promoting Cdh5 interaction with β-catenin (Caradu et al. 2018). The present study suggests that Dhh regulates EC activation and EC junction integrity via distinct pathways. Indeed, while Cdon mainly affects Dhh regulation of endothelial junctions, Gas1 mainly regulates Dhh regulation of EC immune quiescence. In both cases, a dialogue between Ptch1 and Smo seems to be involved, since Cdon modulates Dhh interaction with Ptch1 to regulate EC junctions, while we previously found that Cdh5 junction integrity depends on Smo (Hollier et al. 2020). Similarly, Gas1 promotes Dhh binding with Smo to prevent EC activation, while we found that Ptch1 KD is sufficient to increase VCAM-1 and ICAM-1 expression in ECs. We then hypothesized that both dialogues going from Ptch1 to Smo and Smo to Ptch1 exist based on the reciprocal regulation of Ptch1 and Smo by Smurf family of E3 ubiquitin ligases (Li et al. 2018).

Finally, the main goal of this study was to investigate whether Hh co-receptors may be used to modulate Hh signaling in ECs for therapeutical purposes. By identifying Cdon as a negative regulator of Dhh in ECs, and by demonstrating that Cdon KO prevents BBB opening in the setting of brain inflammation, the present study offers the possibility of using Cdon blocking molecules including blocking antibodies (FIG. 2) as therapeutic tools to preserve endothelial integrity at least in the setting of inflammation. Notably, inflammatory cytokines including TNFα and Il1β increase Cdon expression in ECs.

### REFERENCES

Throughout this application, various references describe the state of the art to which this invention pertains. The disclosures of these references are hereby incorporated by reference into the present disclosure.
Agrawal, V., D. Y. Kim, et Y. G. Kwon. 2017. «Hhip Regulates Tumor-Stroma-Mediated Upregulation of Tumor Angiogenesis». Exp Mol Med 49 (1): e289. https://doi.org/10.1038/emm.2016.139 emm2016139 [pii].
Alvarez, J. I., A. Dodelet-Devillers, H. Kebir, I. Ifergan, P. J. Fabre, S. Terouz, M. Sabbagh, et al. 2011. «The Hedgehog Pathway Promotes Blood-Brain Barrier Integrity and CNS Immune Quiescence». Science 334 (6063): 1727-31.
Argaw, Azeb Tadesse, Blake T. Gurfein, Yueting Zhang, Andleeb Zameer, et Gareth R. John. 2009. «VEGF-Mediated Disruption of Endothelial CLN-5 Promotes Blood-Brain Barrier Breakdown». Proceedings of the National Academy of Sciences of the United States of America 106 (6): 1977-82. https://doi.org/10.1073/pnas.0808698106.
Azzoni, E., V. Conti, L. Campana, A. Dellavalle, R. H. Adams, G. Cossu, et S. Brunelli. 2014. «Hemogenic Endothelium Generates Mesoangioblasts That Contribute to Several Mesodermal Lineages in Vivo». Development 141 (9): 1821-34. https://doi.org/10.1242/dev.103242 141/9/1821 [pii].
Caradu, Caroline, Thierry Couffinhal, Candice Chapouly, Sarah Guimbal, Pierre-Louis Hollier, Eric Ducasse, Alessandra Bura-Rivière, Mathilde Dubois, Alain-Pierre Gadeau, et Marie-Ange Renault. 2018. «Restoring Endothelial Function by Targeting Desert Hedgehog Downstream of Klf2 Improves Critical Limb Ischemia in Adults». Circulation Research 123 (9): 1053-65. https://doi.org/10.1161/CIRCRESAHA.118.313177.
Cardozo, M. J., L. Sanchez-Arrones, A. Sandonis, C. Sanchez-Camacho, G. Gestri, S. W. Wilson, I. Guerrero, et P. Bovolenta. 2014. «Cdon Acts as a Hedgehog Decoy Receptor during Proximal-Distal Patterning of the Optic Vesicle». Nat Commun 5: 4272.
Casillas, Catalina, et Henk Roelink. 2018. «Gain-of-Function Shh Mutants Activate Smo Cell-Autonomously Independent of Ptch1/2 Function». Mechanisms of Development 153: 30-41. https://doi.org/10.1016/j.mod.2018.08.009.
Chapouly, C., Q. Yao, S. Vandierdonck, F. Larrieu-Lahargue, J. N. Mariani, A. P. Gadeau, et M. A. Renault. 2016. «Impaired Hedgehog Signalling-Induced Endothelial Dysfunction Is Sufficient to Induce Neuropathy: Implication in Diabetes». Cardiovasc Res 109 (2): 217-27.
Chapouly, Candice, Sarah Guimbal, Pierre-Louis Hollier, et Marie-Ange Renault. 2019a. «Role of Hedgehog Signaling in Vasculature Development, Differentiation, and Maintenance». International Journal of Molecular Sciences 20 (12). https://doi.org/10.3390/ijms20123076.
—. 2019b. «Role of Hedgehog Signaling in Vasculature Development, Differentiation, and Maintenance». International Journal of Molecular Sciences 20 (12). https://doi.org/10.3390/ijms20123076.
Chinchilla, P., L. Xiao, M. G. Kazanietz, et N. A. Riobo. 2010. «Hedgehog Proteins Activate Pro-Angiogenic Responses in Endothelial Cells through Non-Canonical Signaling Pathways». Cell Cycle 9 (3): 570-79.
Hollier, Pierre-Louis, Candice Chapouly, Aissata Diop, Sarah Guimbal, Lauriane Cornuault, Alain-Pierre Gadeau, et Marie-Ange Renault. 2020. «Endothelial Cell Response to Hedgehog Ligands Depends on Their Processing». BioRxiv, mars, 2020.03.03.974444. https://doi.org/10.1101/2020.03.03.974444.
Horng, S., A. Therattil, S. Moyon, A. Gordon, K. Kim, A. T. Argaw, Y. Hara, et al. 2017. «Astrocytic Tight Junctions Control Inflammatory CNS Lesion Pathogenesis». J Clin Invest 127 (8): 3136-51. https://doi.org/10.1172/JCI91301 91301 [pii].
Jin, S., D. C. Martinelli, X. Zheng, M. Tessier-Lavigne, et C. M. Fan. 2015. «Gas1 Is a Receptor for Sonic Hedgehog to Repel Enteric Axons». Proc Natl Acad Sci USA 112 (1): E73-80. https://doi.org/10.1073/pnas.1418629112 1418629112 [pii].
Kenyon, B. M., E. E. Voest, C. C. Chen, E. Flynn, J. Folkman, et R. J. D'Amato. 1996. «A Model of Angiogenesis in the Mouse Cornea». Invest Ophthalmol Vis Sci 37 (8): 1625-32.
Kogerman, Priit, Darren Krause, Fahimeh Rahnama, Lembi Kogerman, Anne Birgitte Undén, Peter G. Zaphiropoulos, et Rune Toftgård. 2002. «Alternative First Exons of PTCH1 Are Differentially Regulated in Vivo and May Confer Different Functions to the PTCH1 Protein». Oncogene 21 (39): 6007-16. https://doi.org/10.1038/sj.onc.1205865.
Lee, C. S., L. Buttitta, et C. M. Fan. 2001a. «Evidence That the WNT-Inducible Growth Arrest-Specific Gene 1 Encodes an Antagonist of Sonic Hedgehog Signaling in the Somite». Proc Natl Acad Sci USA 98 (20): 11347-52.
—. 2001b. «Evidence That the WNT-Inducible Growth Arrest-Specific Gene 1 Encodes an Antagonist of Sonic Hedgehog Signaling in the Somite». Proceedings of the National Academy of Sciences of the United States of America 98 (20): 11347-52. https://doi.org/10.1073/pnas.201418298.
Li, Shuang, Shuangxi Li, Bing Wang, et Jin Jiang. 2018. «Hedgehog reciprocally controls trafficking of Smo and Ptc through the Smurf family of E3 ubiquitin ligases». Science signaling 11 (516). https://doi.org/10.1126/scisignal.aan8660.
Martinelli, D. C., et C. M. Fan. 2007. «Gas1 Extends the Range of Hedgehog Action by Facilitating Its Signaling». Genes Dev 21 (10): 1231-43.
Nie, D. M., Q. L. Wu, P. Zheng, P. Chen, R. Zhang, B. B. Li, J. Fang, L. H. Xia, et M. Hong. 2016. «Endothelial Microparticles Carrying Hedgehog-Interacting Protein Induce Continuous Endothelial Damage in the Pathogenesis of Acute Graft-versus-Host Disease». Am J Physiol Cell Physiol 310 (10): C821-35. https://doi.org/10.1152/ajpce11.00372.2015 ajpcell.00372.2015 [pii].
Nusslein-Volhard, C., et E. Wieschaus. 1980. «Mutations Affecting Segment Number and Polarity in Drosophila». Nature 287 (5785): 795-801.
Okada, A., F. Charron, S. Morin, D. S. Shin, K. Wong, P. J. Fabre, M. Tessier-Lavigne, et S. K. McConnell. 2006. «Boc Is a Receptor for Sonic Hedgehog in the Guidance of Commissural Axons». Nature 444 (7117): 369-73.
Ramsbottom, Simon A., et Mary E. Pownall. 2016. «Regulation of Hedgehog Signalling Inside and Outside the Cell». Journal of Developmental Biology 4 (3). https://doi.org/10.3390/jdb4030023.
Renault, M. A., J. Roncalli, J. Tongers, T. Thorne, E. Klyachko, S. Misener, O. V. Volpert, et al. 2010. «Sonic Hedgehog Induces Angiogenesis via Rho Kinase-Dependent Signaling in Endothelial Cells». J Mol Cell Cardiol 49 (3): 490-98.
Robbins, D. J., D. L. Fei, et N. A. Riobo. 2012. «The Hedgehog Signal Transduction Network». Sci Signal 5 (246): re6.
Sanchez-Arrones, Luisa, Marcos Cardozo, Francisco Nieto-Lopez, et Paola Bovolenta. 2012. «Cdon and Boc: Two Transmembrane Proteins Implicated in Cell-Cell Communication». The International Journal of Biochemistry & Cell Biology 44 (5): 698-702. https://doi.org/10.1016/j.biocel.2012.01.019.
Sekiguchi, H., M. Ii, K. Jujo, M. A. Renault, T. Thorne, T. Clarke, A. Ito, et al. 2012. «Estradiol Triggers Sonic-hedgehog-induced Angiogenesis During Peripheral Nerve Regeneration by Downregulating Hedgehog-interacting Protein». Lab Invest.
Tenzen, T., B. L. Allen, F. Cole, J. S. Kang, R. S. Krauss, et A. P. McMahon. 2006. «The Cell Surface Membrane Proteins Cdo and Boc Are Components and Targets of the Hedgehog Signaling Pathway and Feedback Network in Mice». Dev Cell 10 (5): 647-56.
Xia, Y. P., Q. W. He, Y. N. Li, S. C. Chen, M. Huang, Y. Wang, Y. Gao, et al. 2013. «Recombinant Human Sonic Hedgehog Protein Regulates the Expression of ZO-1 and Occludin by Activating Angiopoietin-1 in Stroke Damage». PLoS One 8 (7): e68891.
Zhang, Wei, Jong-Sun Kang, Francesca Cole, Min-Jeong Yi, et Robert S. Krauss. 2006. «Cdo Functions at Multiple Points in the Sonic Hedgehog Pathway, and Cdo-Deficient Mice Accurately Model Human Holoprosencephaly». Developmental Cell 10 (5): 657-65. https://doi.org/10.1016/j.devce1.2006.04.005.

1. A method of treating endothelial dysfunction in a patient in need thereof comprising administering to the patient a therapeutically effective amount of a Cdon inhibitor.

2. The method of claim 1 wherein the patient suffers from a systemic inflammatory response syndrome or sepsis.

3. The method of claim 1 wherein the patient suffers diabetes mellitus.

4. The method of claim 1 wherein the patient suffers from diabetic micro- and/or macroangiopathy.

5. The method of claim 1 wherein the patient suffers from diabetic nephropathy, diabetic dermopathy, diabetic retinopathy and diabetic neuropathy.

6. The method of claim 1 wherein the patient suffers from peripheral arterial disease.

7. The method of claim 6 wherein the peripheral arterial disease is selected from the group consisting of acute and chronic critical limb ischemia, Buerger's disease and critical limb ischemia in diabetes.

8. The method of claim 1 wherein the Cdon inhibitor is an antibody having binding affinity for Cdon.

9. The method of claim 1 wherein the Cdon inhibitor is an antibody directed against the extracellular domain of Cdon.

10. The method of claim 1 wherein the Cdon inhibitor is an antibody having binding affinity for the region of Cdon which binds to Dhh.

11. The method of claim 10 wherein the Cdon inhibitor is an antibody that binds to Fibronectin type-III 3 domain of Cdon.

12. The method of claim 1 wherein the Cdon inhibitor is an antibody having binding affinity for the amino acid sequence ranging from the amino acid residue at position 826 to the amino acid residue at position 926 in SEQ ID NO: 1.

13. The method of claim 1 wherein the Cdon inhibitor is an inhibitor of expression that directly blocks the translation of Cdon mRNA by binding thereto and thus preventing protein translation or increasing mRNA degradation.

14. The method of claim 13 wherein the inhibitor of expression is a siRNA, an antisense oligonucleotide or a ribozyme.

15. The method of claim 13 wherein the inhibitor of expression is an endonuclease.
